(19) United States
(12) Patent Application Publication
(10) Pub. No.: US 2009/0285921 A1
(43) Pub. Date: Nov. 19, 2009

(54) PLANT EXTRACT AND ITS THERAPEUTIC USE

(75) Inventor: Matthias-Heinrich Kreuter, Walenstadt (CH)

Correspondence Address: BUCHANAN, INGERSOLL & ROONEY PC, POST OFFICE BOX 1404, ALEXANDRIA, VA, 22313-1404, US

(73) Assignee: Veritron Limited, London (GB)

(21) Appl. No.: 12/325,811

(22) Filed: Dec. 1, 2008

(30) Foreign Application Priority Data
May 16, 2008 (GB) 0808974.0

(51) Int. Cl. A61K 36/28

(52) U.S. Cl. 424/764

(57) ABSTRACT

The present invention is directed to a composition comprising an aqueous extract of camomile flowers for the treatment of a proliferative and/or viral condition.

### FIELD OF THE INVENTION

This invention relates to a plant extract and its therapeutic use.

### BACKGROUND OF THE INVENTION

The therapeutic properties of various plants have been known for millennia. Even today, however, the nature of the effective component or components and their properties are little understood, even for those plants that have been studied, since pharmaceutical development generally focuses on small molecules that are deemed to have relatively predictable properties and whose synthesis can be controlled.

Uteshev et al, Eksp. Klin. Farmakol. (1999 November-December) 62(6):52-5, describes the immunomodulating activity of heteropolysaccharides obtained from German camomile (Matricaria chamomilla) during air and immersion cooling. Laskova and Uteshev, Antibiot. Khimioter. (1992 June) 37(6):15-8, describes the immunomodulating action of heteropolysaccharides isolated from camomile flowers. The water-based extract was administered orally or by intraperitoneal injection. The authors do not suggest any therapeutic utility, but rather report that the stimulatory effect is dependent on dosing regime and, primarily, the manner and degree of cooling of the tested rats.

WO03/101479 describes the valuable therapeutic properties of a composition comprising various components, typically given together by intramuscular injection. The composition that was used in the examples comprises a camomile extract, although no therapeutic activity is ascribed to it; rather, it is described as an anti-irritant whose presence may alleviate the unpleasant effect of the injection per se.

WO2007/057651 discloses a process for the removal of endotoxins from camomile.

### SUMMARY OF THE INVENTION

Surprisingly, it has now been found that a camomile extract, obtained from the flower heads, such as that described in WO2007/057651 has valuable therapeutic properties. In particular, it has been found that it can reduce DNA and RNA synthesis without substantial effect on protein synthesis, from which utility in the treatment of cancer may be deduced.

### DESCRIPTION OF THE INVENTION

The invention is based on data obtained using an aqueous extract of camomile. It is possible that the active component or components may be found in other plants. Their suitability can readily be tested by one of ordinary skill in the art, e.g. on the basis of the evidence provided below, for the activity of a camomile extract. Suitable herbs and other plants may already be known. Examples include Matricaria recutita L. (Asteraceae), Calendula officinalis L. (Asteraceae), Achillea millefolium L. (Asteraceae) and Youngia japonica L. (Asteraceae).

The available evidence shows that the camomile extract obtained as described below maintains protein biosynthesis, whereas DNA- and RNA-biosynthesis is reduced. This is a good measure of the desirable properties of this extract.

The extract may be obtained by any suitable procedure, including methods known to those of ordinary skill in the art. The extract may be obtained by using an aqueous or organic medium, and separated from other components by filtration, chromatography etc. For example, a material that may be used in the invention is derived from the dried flower heads of the composite plant Matricaria recutita L. or one or more materials therein, including polysaccharides, glycoproteins, volatile oils, chamazulene, anthemic acid, apigenine, glycosides and other substances.

A preferred extraction procedure is described in WO2007/057651.

Purification or purifying according to the present invention can be conducted on the whole composition or an extract or a fraction of an extract.

According to that process, an aqueous composition comprising a water-soluble contaminant having lipid groups, is contacted with a lipophilic component that forms a complex with the contaminant; there then follow a first removal step, of material having a size larger than the complex, and a second removal step, of the complex.

In the process, the second removal step is typically ultrafiltration, and removes the endotoxins that complex with the lipophilic component. The first filtration or other removal step is necessary to remove larger components that otherwise will block the ultrafiltration process.

Preferably, the extract contains at least one water-soluble contaminant having lipid groups, and purifying comprises or the process further comprises the steps of:
(i) contacting the composition with a lipophilic component that forms a complex with the contaminant;
(ii) a first removal step of removing material having a particle size larger than the complex formed in step (i); and
(iii) a second removal step of removing the complex formed in step (i).

More preferably, the contaminant is an endotoxin, preferably selected from the group consisting of fragments of the cell wall or fragments of molecules constituting the cell wall of Gram negative bacteria, more preferably selected from the group consisting of lipopolysaccharides and carbohydrates having protein groups, in particular glycoproteins having at least one lipid chain.

Still more preferably, the endotoxin is a carbohydrate having protein groups, in particular a glycoprotein having at least one lipid chain.

In a preferred embodiment, the endotoxin has a molecular weight in excess of 10,000 dalton, more preferably in excess of 5000 dalton, yet more preferably in excess of 1000 dalton and most preferably in excess of 500 dalton.

In another preferred embodiment, the lipophilic component is an oil, preferably a fatty oil.

In a further preferred embodiment, step (ii) comprises microfiltration, preferably using a filter having a pore size of at least 0.05 μm, more preferably having a pore size of from 0.05 to 0.2 μm, most preferably of 0.1 μm.

In a preferred embodiment, step (iii) comprises ultrafiltration, preferably using a filter having a pore size of from 0.001 to 0.02 μm, more preferably from 0.001 to 0.01 μm.

In a preferred embodiment, the extract is free or essentially free of endotoxins, preferably the extract contains endotoxins in an amount of 100 EU/ml (endotoxin units per ml according to Ph. Eur.) or less, more preferably 75 EU/ml or less, yet more preferably 50 EU/ml or less, still yet more preferably 25 EU/ml or less, and most preferably 20 EU/ml or less.

In a preferred embodiment, the extract is free or essentially free of compounds selected from the group consisting of apigenine, the glycosides of apigenine and essential oils, more preferably free or essentially free of apigenine, the glycosides of apigenine and essential oils, most preferably the extract contains apigenine, the glycosides of apigenine and essential oils in an amount of 20 ppm or less, in particular 100 ppb or less.

Preferably, the composition additionally comprises ascorbic acid or a salt thereof.

Preferably, the composition additionally comprises at least one selected from the group consisting of pharmaceutical aids, preferably selected from the group consisting of pharmaceutical agents and pharmaceutical excipients.

The lipophilic component used in the present invention can be the same as that described in U.S. Pat. No. 6,024,998. Whereas such a component can form relatively large drops of a lipophilic phase in which lipophilic contaminants are dissolved, a characteristic of the present invention is that such a material can also complex with lipid groups in a generally water-soluble molecule such as an endotoxin; the complex is of a size that can be removed by ultrafiltration but not by microfiltration that is sufficient to remove the drops. Therefore, while the materials used in this invention may be the same as those in the prior art, the procedure is necessarily different.

Endotoxins and also antigens are primarily carbohydrates having pendant protein and lipid groups; the presence of the lipid groups is sufficient to form a 15 complex with a suitable lipophilic material, but does not compromise the generally water-soluble nature of the carbohydrate molecule. Such pyrogenic molecules may have an inflammatory effect, on injection, and they should therefore be removed as far as possible from an injectable medicament.

The flower head (capitulum) of the camomile plant (Matricaria recutita) is composed of two parts, i.e. the yellow disc-shaped or tubular flowers or florets (flores tubiformis or tubiflorum) and the white radiating flowers or florets (flores ligutatea). The former is of particular interest. By means of the process described herein, a useful product can be obtained by separating the tubular flowers from other parts of the camomile head/plant, extraction of the separated yellow part in water, and isolation of the extract/removal of endotoxins.

Lipophilic components suitable for use in the invention are described in U.S. Pat. No. 6,024,998. This component may be of animal, vegetable, mineral or synthetic origin. It is preferably non-toxic. Examples of suitable materials include fats such as cocoa butter and coconut fat; oils such as neutral oils, sunflower oils, and fractionated coconut oil; waxes such as stearins, jojoba oil, beeswax, spermaceti and carnauba wax; paraffins, including vaseline; lipids; and sterols. All such compounds, whether pure or used as mixtures, preferably meet the requirements of the Deutsches Arzneibuch, the British Pharmacopoeia, the European Pharmacopeia or the US Food Chemical Codex. Particularly preferred materials are miglyol, diglycerides, triglycerides and ricinus oil. This last material includes ricinoleic acid, an example of a long-chain fatty acid containing a polar group.

The aqueous extract that may be subjected to a purification process according to the present invention typically comprises a multi-component mixture of water-soluble components. It may be obtained by adding water to the appropriate plant part, to obtain a suspension that is then usually heated to a temperature below the boiling point of water, e.g. 90-94° C., and then cooled to room temperature.

The aqueous extract is then subjected to the two filtration steps. For the purposes of illustration only, these will be described below as microfiltration and ultrafiltration, respectively. Other techniques, such as use of a lipophilic barrier, may be suitable. Each filtration step may be conducted in one, two or more than two stages, if desired.

As indicated above, microfiltration is applied in order to remove material that would otherwise compromise the effectiveness of the ultrafiltration step. Microfiltration may indeed remove contaminants, as described in U.S. Pat. No. 6,024,998. This typically involves using a filter having a pore size of at least 0.1 μm. The pore size used in the subsequent, ultrafiltration step is typically 0.001 to 0.01, e.g. up to 0.1, μm.

Each filtration step is preferably conducted by membrane separation, using synthetic membranes of materials such as glass, metal, ceramic or synthetic plastics. Materials suitable for microfiltration include polypropylene and polytetrafluorethylene. Materials suitable for ultrafiltration include polyether sulfones and regenerated cellulose.

When two liquid phases are separated, this is preferably conducted by means of membrane technology. For this purpose, tubular or so-called “cross-flow” membranes are preferred.

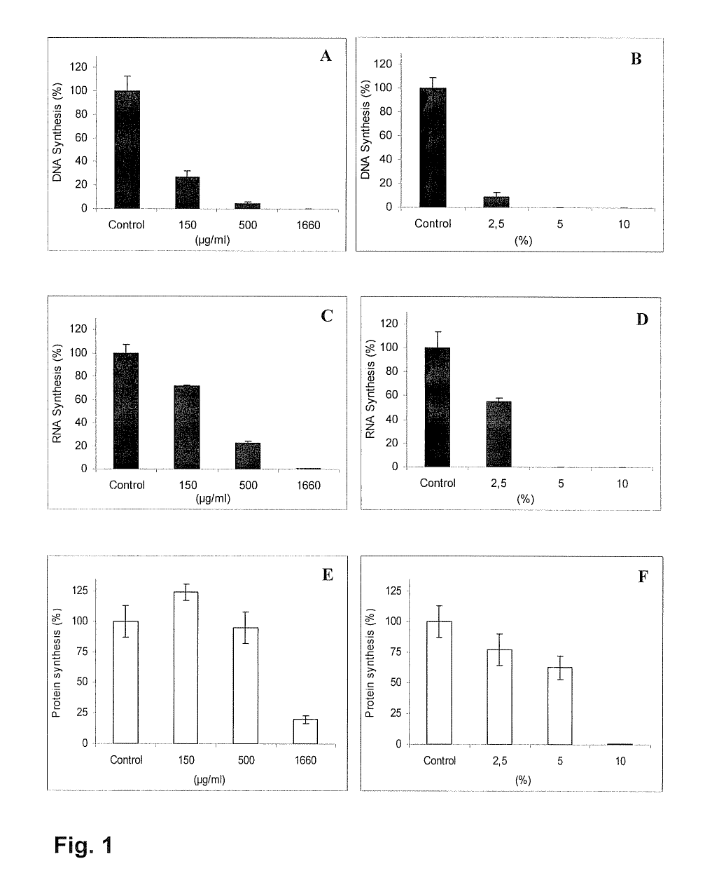

The product is intended for use in therapy. It should then be sterile, and it is desirable that appropriate steps of its production should be conducted under sterile conditions. Such steps are these shown as 19, 21, 23 and 26, in FIG. 1B of WO2007/057651. Such a procedure is illustrated in the following Examples 1 to 5. Example 6 also illustrates the process, using a revised protocol.

The experimental work reported in the examples shows that the combination of a filtration cascade and the addition of a plant oil leads to a complete or nearly complete elimination of bacterial cell wall debris, known to a person skilled in the art as bacterial endotoxins or pyrogenes. These lipopolysaccharides or macromolecules are composed of a Lipid A moiety attached to a polysaccharide chain and are a major constituent of the cell wall of gram-negative bacteria. These complex macromolecules are water-soluble but surprisingly form high molecular complexes with plant oils resulting in a suspension and can be retained by molecular weight exclusion techniques, preferably by using ultrafiltration equipment. Molecular weight filtration microfiltration is of advantage to get rid of large piece of cell wall debris, mucilaginous cell wall fragments of the plant materials which would otherwise block the pores of ultrafiltration equipment.

The analysis of bacterial endotoxins of the samples obtained in the Examples was performed with the Cambrex PyroGene assay using a dilution factor of 1:10.000.

### EXAMPLES 1 AND 2

45 g of yellow tubular camomile flowers (Chamomilla recutita) were mixed with 900 g of water (Aqua purificata, Ph. Helv.) This mixture was heated to a temperature between 90° C. and 94° C. within 20 to 30 minutes. Thereafter the mixture was stored at room temperature (15° C. to 25° C.) until a temperature between 30° C. and 35° C. was reached.

The drug residue was removed by deep layer filtration. The obtained crude filtrate was clarified by filtration through a 0.22 μm membrane.

To the clarified filtrate, 0.3% (Example 1) or 0.1% (Example 2), with respect to the extract mass, of ricinus oil (Ph. Eur. Grade) was added. The whole mixture was homogenised for 5 minutes. This prepared extract was filtered (in tangential flow mode) with retentate recovery via a 0.22 μm membrane.

The obtained permeate was filtered (in tangential flow mode) with retentate recovery via a 0.1 μm membrane. Finally, the obtained permeate was filtered (in tangential flow mode) with retentate recovery via a 1000 kDa membrane. Residue of bacterial endotoxins in each final filtrate: <100 EU/ml

### EXAMPLES 3 TO 5

Example 1 was repeated, except that, instead of ricinus oil, 0.3% (Example 3), 1.0% (Example 4) and 3.0% (Example 5), with respect to the extract mass, of mygliol (Ph. Eur.) was added to the clarified filtrate. Residue of bacterial endotoxins in each final filtrate: <100 EU/ml.

### EXAMPLE 6

This Example uses a revised protocol, in which heating and cooling were performed, not in an autoclave but in a 10 L double layer vessel under stirring (max. temperature of heating device 140° C.).

Mycliol was added instead of ricinus oil. The miglyol was “Miglyol 812 for parenteral use” from Hanseler. The mixture was stirred at room temperature for 10 minutes, instead of homogenization.

Microfiltrations according to the earlier process were all performed with Millipore Pellicon 2 systems. For better practicability and to avoid time-consuming cleaning procedures, the microfiltrations in this Example were performed with the following equipment:

| Filtration | Filter System | Cartouche |
|---|---|---|
| 0.2 μm filtration | Millipore Pellicon 2 | Durapore 0.2μ, C-screen |
| 0.1 μm filtration | One way filter | Millipack 200, 0.1 μm |
| 1000 kDa filtration | Millipore Pellicon 2 | Biomax 1000 kDa, V-Screen |
| 0.2 μm filtration | One way filter | Millipack 200, 0.2 μm |

In addition, phenol was added, for stabilization of the extract. The amount of added phenol was 6.0-8.0 mg/ml. It was added after the 1000 kDa filtration. After the addition, the suspension was stirred for approximately 10 minutes, until all phenol was dissolved.

The endotoxin level was low in each case.

A composition of the invention may comprise, if desired, one or more of the other components described in WO03/101479 (the content of which is herein incorporated by reference). Such components include vitamins such as vitamin C or ascorbic acid (or a salt thereof) and vitamin B complexes, metal salts that provide metal ions in vivo such as Ca ions, insulin and/or an antihistamine. The desirability of using any such additional component can readily be determined by one ordinarily skilled in the art. Ascorbic acid may be present in the extract.

The source of the camomile extract is important. It should be the flower head.

The composition that is used should be suitable for injection. For this purpose, it is desirable to remove endotoxins and (by any suitable means, known to those in the art) large molecular weight component, e.g. those having a m. wt. of more than 500, 1,000, 5,000 or 10,000.

Compositions for use in the invention can be formulated by methods known to those skilled in the art. Pharmaceutically acceptable components should be used. The term “pharmaceutically acceptable” refers to those properties and/or substances which are acceptable to the patient from a pharmacological/toxicological point of view and to the manufacturing pharmaceutical chemist from a physical/chemical point of view regarding factors such as formulation, stability, patient acceptance and bioavailability. Administration is preferably by intravenous or, more preferably, intramuscular injection.

The pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients such as, for example, inert diluents such as calcium carbonate, sodium carbonate, lactose, calcium phosphate or sodium phosphate; granulating and disintegrating agents, for example corn starch or alginic acid; binding agents, for example starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glycerol monostearate or glycerol distearate may be employed. They may also be coated, to form osmotic therapeutic tablets for control release.

Formulations for oral use may also be presented as hard gelatine capsules wherein the active ingredient is mixed with an inert solid diluent, for example calcium carbonate, calcium phosphate or kaolin, or as soft gelatine in capsules wherein the active ingredient is mixed with water or an oil medium, for example peanut oil, liquid paraffin or olive oil.

Aqueous suspensions may contain the active materials in admixture with suitable excipients. Such excipients are suspending agents, for example sodium carboxymethylcellulose, methylcellulose, hydroxypropylmethylcellulose, sodium alginate, polyvinyl pyrrolidone, gum tragacanth and gum acacia; dispersing or wetting agents, for example a naturally occurring phosphatide such as lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol, such as a polyoxyethylene with partial esters derived from fatty acids and hexitol anhydrides, for example polyoxyethylene sorbitan monooleate. Aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents (such as those set forth above) and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified above. Sweetening, flavouring and colouring agents may also be present.

A pharmaceutical composition for use in the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulated using suitable dispersing or wetting agents and suspending agents, examples of which have been mentioned above. A sterile injectable preparation may also be in a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The composition may also be administered in the form of suppositories for rectal administration of the drug. Such compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, suitable compositions are in the form of, for example, creams, ointments, jellies, solutions or suspensions. For the purposes of this specification, topical application includes mouth washes and gargles.

As indicated above, composition of the invention may be given by injection. Intramuscular injection is preferred, although any parenteral administration is suitable.

It may also be preferred that the composition is given orally. In this case, and in the event that the permeability-increasing agent is used, insulin should not be included in an oral formulation. Oral administration may be particularly preferred for veterinary medicine.

Other active materials may also be given to the subject. Although it is not believed that further materials are necessary, it has been found that certain steroids and vitamins, typically given orally, can support or enhance the effect of the medicament. Suitable steroid hormones may increase the synthesis of specific proteins, by unmasking certain cistrons, with the assistance of essential metabolites such as vitamins and amino acids. Examples of suitable steroids are estradiol, nandrolone and estriol. Vitamins such as A, D and/or E may also be given. The function of vitamin A may be to preserve the integrity of epithelial tissue, to play a role in protein synthesis, and to stabilise cell membranes and also subcellular membranes.

Therapeutic use according to the invention involves the treatment (and possibly also the prevention) of cancer, especially liver cancer, e.g. tumours comprising HEPG2 cells. These and others include topical conditions such as psoriasis, scleroderma and pemphigus, infectious bronchitis, cancers including sarcomas (such as Kaposi's sarcoma), leukemia, skin cancer and the carcinomas whose treatment is specifically illustrated below, as well as AIDS. For the treatment of leukemia, the composition may be supplemented by the administration of a growth hormone. More generally, it may be used for therapy of proliferative and viral conditions, especially those associated with DNA or RNA viruses. The action on RNA viruses may be direct, while the action on DNA viruses and cancer at least may be progressive. The medicament may also be useful in therapy of other genetic disorders such as motor neurone disease and multiple sclerosis.

In the treatment of cancer, it has been found that the medicament may promote the patient's quality of life, without necessarily reducing tumour size. For example, it facilitates a healing process that has the following stages:
(i) metastasis and tumour growth cease (and the tumour mass may begin to decrease); and
(ii) the tumour is converted to a hard consistency by a process of fibrosis, and is isolated from surrounding normal tissue, which allows the tumour to be easily removed by surgery.

It appears that the medicament affects the immune system indirectly, by normalising body cells. It apparently has both direct and indirect effects in its anti-viral activity, demonstrated by its very rapid direct action on RNA viruses and by its direct and indirect action on DNA viruses and cancers. It apparently normalises otherwise abnormal genetic material; the effect may be both chemical and functional. It also appears that most of the cellular components become targets of the medicament, especially cell surface receptors and genetic material. Despite the effect on cells, no adverse side-effects have been detected, even after treatment for more than 18 months in cancer patients.

For the treatment of AIDS, the patient may have the human retrovirus (HRV); this indicates human immunodeficiency virus type 1, or strains thereof apparent to one skilled in the art, which belong to the same viral families and which create similar physiological effects in humans as various human retroviruses.

Patients to be treated include those individuals (1) infected with one or more strains of a human retrovirus, as determined by the presence of either measurable viral antibody or antigen in the serum, and (2) having either a symptomatic AIDS-defining infection such as (a) disseminated histoplasmosis, (b) isopsoriasis, (c) bronchial and pulmonary candidiasis including pneumocystic pneumonia, (d) non-Hodgkin's lymphoma or (e) Kaposi's sarcoma or having an absolute CD4 lymphocyte count of less than 200/mm³ in the peripheral blood.

Patients who are HIV-positive but asymptomatic would typically be treated with lower doses. ARC (AIDS-related complex) and AIDS patients would typically be treated with higher doses.

As may possibly be desired, components used in this invention can be administered in conjunction with (or simultaneously, concomitantly or sequentially with) other antiviral agents such as AZT, ddl, ddC, 3TC, d4T or non-nucleoside anti-AIDS agents.

Treatment of AIDS, as described herein, refers to inhibition of the HIV virus and will vary, depending on the infected individual. For individuals who are HIV-positive (infected) but who are asymptomatic, the medicament may delay or prevent the onset of symptoms. For individuals who are HIV-positive and symptomatic, and are pre-AIDS or ARC patients, the medicament may delay or prevent the onset of “full-blown AIDS”. For individuals who have “full-blown AIDS”, the medicament may at least considerably extend the survival time of these individuals.

The medicament can also be used to treat other viral conditions. For example, the virus may be a coronavirus, as in the case of SARS (severe acute respiratory syndrome). Further, as indicated above, it may have utility in veterinary medicine, e.g. in fowl's diseases such as Newcastle disease and fowlpox.

Although some indication has been given as to suitable dosages of certain materials, the exact dosage and frequency of administration depend on several factors. These factors include the particular components that are used, the particular condition being treated, the severity of the condition, the age, weight and general physical condition of the particular patient, and other medication the individual may be taking, as is well known to those skilled in the art.

The following study illustrates the invention. The study that is reported uses “CHARE006” which is the camomile extract obtained in Example 4, using hot water extraction as described and purification with 1% mygliol. CHARE006 was also used as a component of “AMT” (the composition otherwise as described in WO03/101479.)

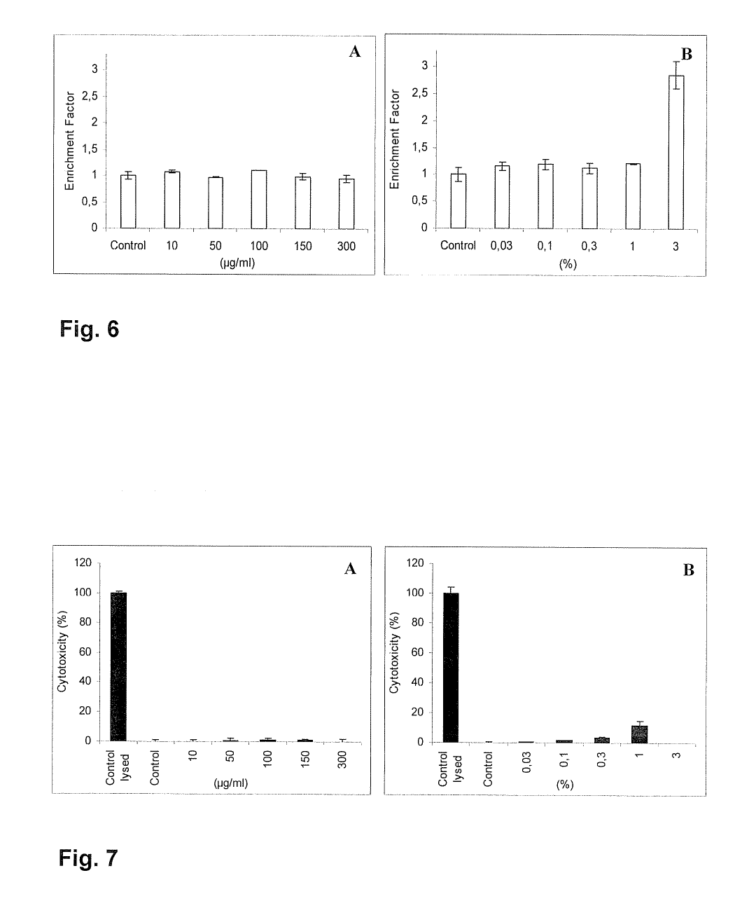

Results are shown in the accompanying drawings. FIGS. 1 to 5 relate to System 4; FIGS. 6 to 8 relate to System 5.

In the following part is given a short description of the Figures:

FIG. 1 relates to System 4 and shows the effect of the extract according to example 4 (VIP-E_Matr'06_{—}1003) on the DNA synthesis (A), on the RNA synthesis (C), and on the protein biosynthesis (E) as well as the effect of AMT (AMT 2003) on the DNA synthesis (B), on the RNA synthesis (D), and on the protein biosynthesis (F) in HepG2 cells. The used extract concentrations are 150, 500, and 1660 μg/ml. The used AMT concentrations are 2.5, 5, and 10%.

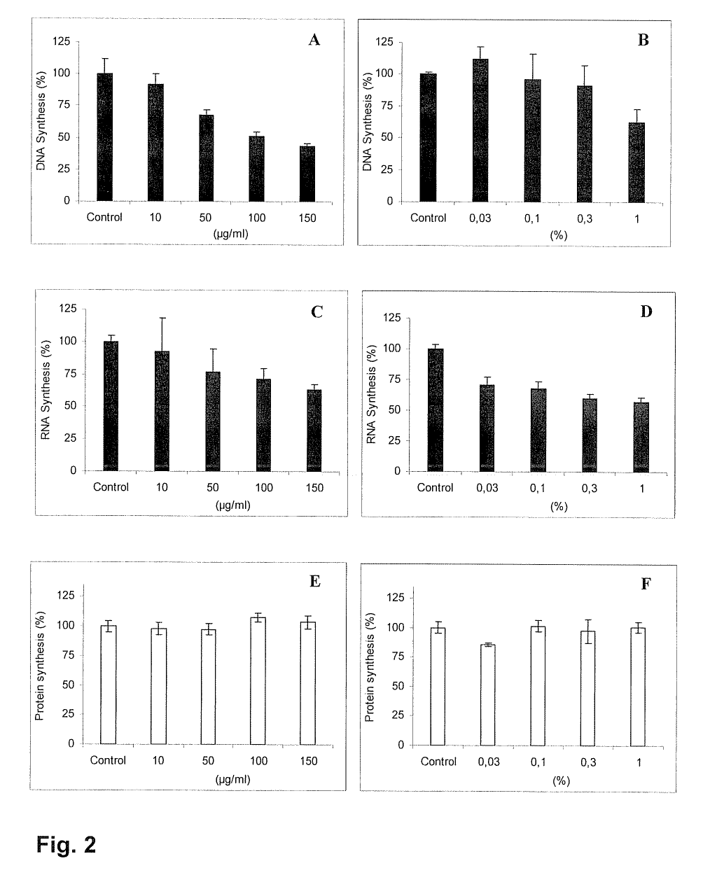

FIG. 2 is in analogy to FIG. 1, but the used extract concentrations are 10, 50, 100, and 150 μg/ml and the used AMT concentrations are 0.03, 0.1, 0.3, and 1%.

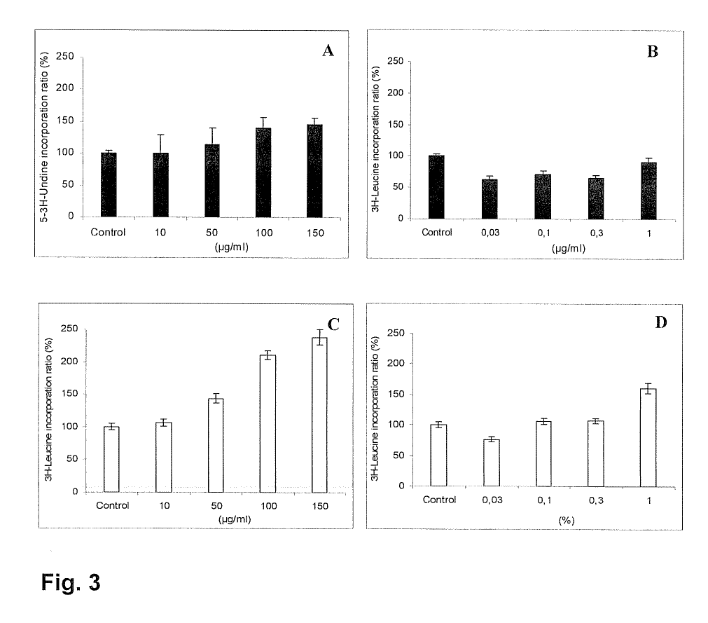

FIG. 3 relates to System 4 and shows the effect of the extract according to example 4 on RNA synthesis (A) and protein biosynthesis (C) as well as the effect of AMT on RNA synthesis (B) and protein biosynthesis (D). The results are calculated in consideration of the values as shown in FIG. 2(A) and FIG. 2(B), respectively.

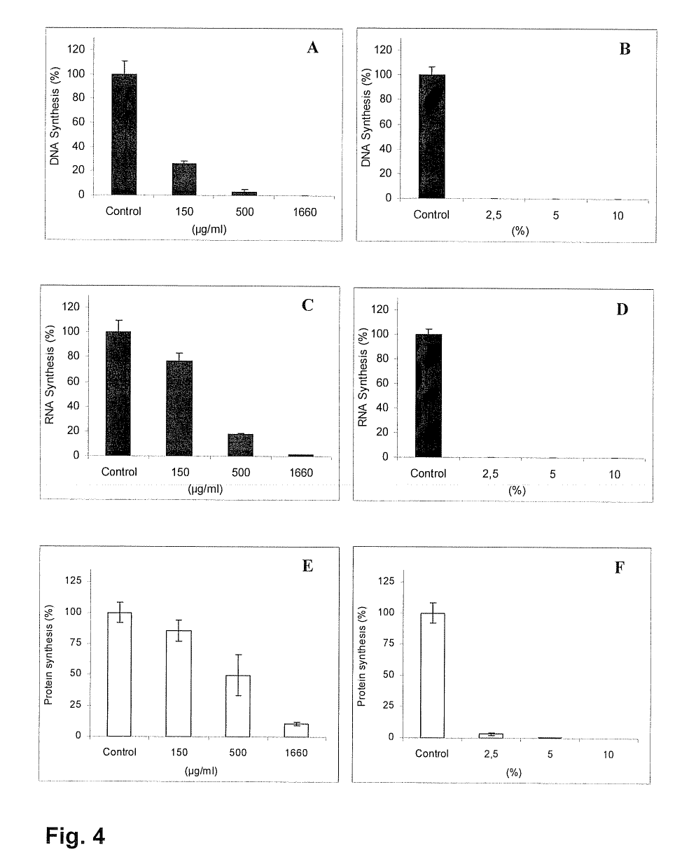

FIG. 4 relates to System 4 and shows the effect of the extract according to example 4 (VIP-E_Matr'06_{—}1003) on the DNA synthesis (A), on the RNA synthesis (C), and on the protein biosynthesis (E) as well as the effect of AMT (AMT 2003) on the DNA synthesis (B), on the RNA synthesis (D), and on the protein biosynthesis (F) in HT1376 cells. The used extract concentrations are 150, 500, and 1660 μg/ml. The used AMT concentrations are 2.5, 5, and 10%.

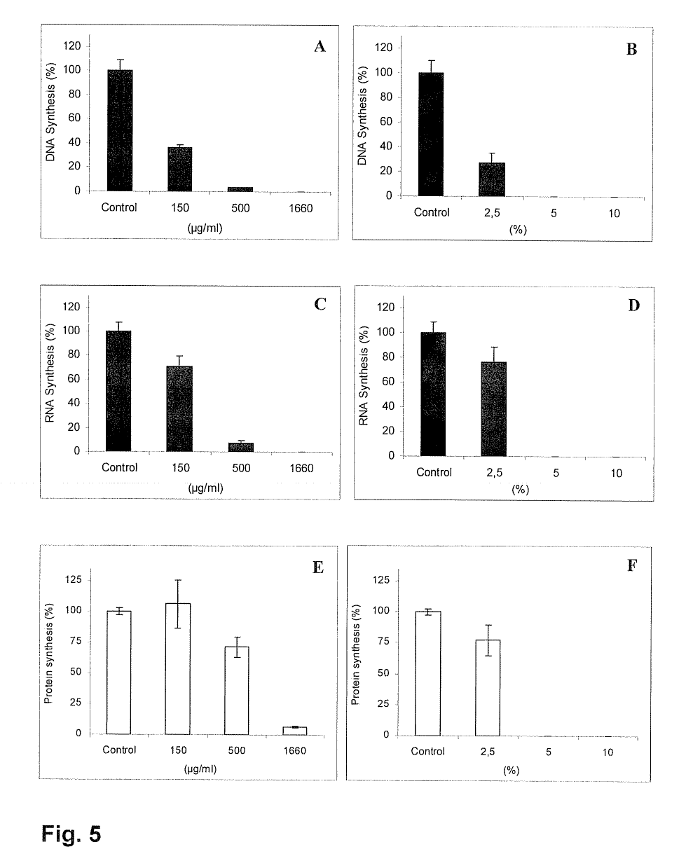

FIG. 5 relates to System 4 and shows the effect of the extract according to example 4 (VIP-E_Matr'06_{—}1003) on the DNA synthesis (A), on the RNA synthesis (C), and on the protein biosynthesis (E) as well as the effect of AMT (AMT 2003) on the DNA synthesis (B), on the RNA synthesis (D), and on the protein biosynthesis (F) in C33-A cells. The used extract concentrations are 150, 500, and 1660 μg/ml. The used AMT concentrations are 2.5, 5, and 10%.

FIG. 6A relates to System 5 and shows the effect of the extract according to example 4 (VIP-E_Matr'06_{—}1003) on the induction of the apoptosis in HepG2 cells after 24 hours. The used extract concentrations are 10, 50, 100, 150, and 300 μg/ml.

FIG. 6B relates to System 5 and shows the effect of AMT (AMT 2003) on the induction of the apoptosis in HepG2 cells after 24 hours. The used AMT concentrations are 0.03, 0.1, 0.3,1, and 3%.

FIG. 7A relates to System 5 and shows the effect of the extract according to example 4 (VIP-E_Matr'06_{—}1003) on the cytotoxicity (membrane integrity) in HepG2 cells after 24 hours. The used extract concentrations are 10, 50, 100, 150, and 300 μg/ml.

FIG. 7B relates to System 5 and shows the effect of AMT (AMT 2003) on the cytotoxicity (membrane integrity) in HepG2 cells after 24 hours. The used AMT concentrations are 0.03, 0.1, 0.3,1, and 3%.

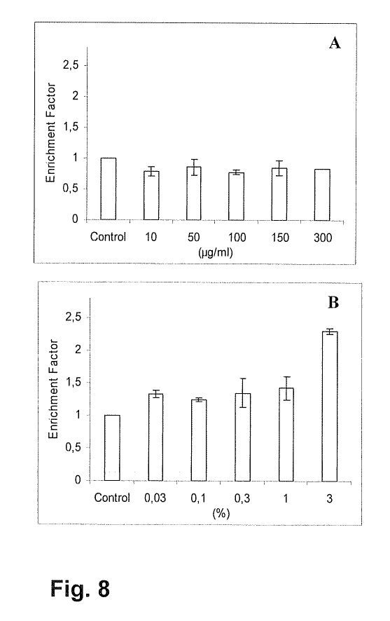

FIG. 8A relates to System 5 and shows the effect of the extract according to example 4 (VIP-E_Matr'06_{—}1003) on the induction of the apoptosis in HepG2 cells after 48 hours. The used extract concentrations are 10, 50, 100, 150, and 300 μg/ml.

FIG. 8B relates to System 5 and shows the effect of AMT (AMT 2003) on the induction of the apoptosis in HepG2 cells after 48 hours. The used AMT concentrations are 0.03, 0.1, 0.3,1, and 3%.

### 1) AIM OF THE STUDY

### System 4: RNA-, DNA- and Protein Biosynthesis

The influence of CHARE 006 and AMT on RNA-, DNA- and protein biosynthesis using three different cell lines (HepG2: liver carcinoma cells; C33-A: cervix carcinoma cells and HT1376: bladder carcinoma cells) was examined in vitro.

### System 5: Apoptosis and Membrane Integrity

Additionally the influence of CHARE 006 and AMT on the induction of Apoptosis was tested on HepG2 cells. At the same time the cytotoxicity (Membrane integrity) of CHARE 006 and AMT was examined on HepG2 cells.

### 2) MATERIAL AND METHODS

### 2.1) Samples and Sample Preparation

| Extract | Description | Origin | Lot | Extract Type |
|---|---|---|---|---|
| VIP-E_Matr′06_1003 | Matricaria recutita | Pentapharm | 578-01/End; | Aqueous |
| | tubular flowers | | Jul. 20, 2006 | |

| Sample | Components | Product No: | Lot | Content |
|---|---|---|---|---|
| AMT | AMT 2003 Vit(+) | 76358131 | 72882G003 | Chlorpheniramine |
| 2003 | (AMT Vitamine ampulls) | | | solution |
| | | | | (with antioxidant) |
| | AMT 2003 Cal. | 76395011 | 0602000268-2 | Buffer salts |
| | (AMT Calcium Sandoz | | | |
| | Inj.) | | | |
| | AMT 2003 Ins. | 76358112 | 0508000342 | Human insulin |
| | (AMT Actrapid Penfill) | | | solution |
| | AMT 2003 Cam. | | 578-01/End; | Chamomile extract |
| | (VIP-E_Matr′06_1003) | | Jul. 20, 2006 | (with phenol) |

10 ml of AMT 2003 stock solution was freshly prepared before use: 1.8 ml of AMT 2003 Cam. (VIP-E_Matr'06_{—}1003), 4.25 ml of AMT 2003 Vit(+), 0.25 ml of AMT 2003 Ins. and 3.2 ml of AMT 2003 Cal were mixed. All samples were diluted in H₂O_{ultra pure} and treated sterile.

### 2.2) Assay Conditions

| | | Incu- | | |
|---|---|---|---|---|
| | Cell | bation | | Sample |
| Assay | lines | time | Sample | concentration |
| RNA | HepG2 | 24 h | VIP- | 150/500/1660 μg/ml |
| Synthesis | C33-A | | E_Matr′06_1003 | 10/50/100/ |
| | HT1376 | | | 150 μg/ml |
| | | | AMT 2003 | 2.5/5/10% |
| | | | | 0.03/0.1/0.3/1% |
| DNA | HepG2 | 24 h | VIP- | 150/500/1660 μg/ml |
| Synthesis | C33-A | | E_Matr′06_1003 | |
| | HT1376 | | AMT 2003 | 2.5/5/10% |
| Protein | HepG2 | 24 h | VIP- | 150/500/1660 μg/ml |
| biosynthesis | C33-A | | E_Matr′06_1003 | |
| | HT1376 | | AMT 2003 | 2.5/5/10% |
| Apoptosis | HepG2 | 24 h | VIP- | 10/50/100/150/ |
| | | | E_Matr′06_1003 | 300 μg/ml |
| | | | AMT 2003 | 0.03/0.1/0.3/1/3% |
| Cytotoxicity | HepG2 | 24 h | VIP- | 10, 50, 100, 150, |
| (Membrane | | | E_Matr′06_1003 | 300 μg/ml |
| integrity) | | | AMT 2003 | 0.03/0.1/0.3/1/3% |

### 2.3) Assays

### 2.3.3) RNA-/DNA Synthesis

For the RNA- and DNA synthesis, assay cells (HepG2: hepatocellular carcinoma, human; C33-A: cervical carcinoma, human and HT1376: bladder carcinoma, human) were harvested by trypsinisation and seeded at 10,000 cells/well in a 96 well plate. After treatment of the cells with the samples at the required concentrations the plate was incubated for 2 hours at 37° C. and 5% CO₂. The cells were pulsed with 5-³H-Uridine (1 μCi/ml) (Perkin Elmer) for the RNA synthesis and with 6-³H-Thymidine (1 μCi/ml) (Perkin Elmer) for the DNA synthesis during a further incubation period of 24 hours. The cells were washed with PBS and fixed twice with methanol for 5 min. The protein was precipitated by 0.3N TCA. After a washing step 150 μl 0,3N NaOH was added for 15 min to lyse the cells. Negative controls t(0) were carried out with the samples without cells.

To detect the incorporated 5-³H-Uridine for the RNA synthesis and the 6-³H-Thymidine for the DNA synthesis the samples were transferred in scintillation tubes with scintillation cocktail. The quantification was performed in a Tri-Carb 1900 TR liquid scintillation counter (Packard, USA).

The effect of several concentrations of samples was measured by determining amount of radiolabel (dpm) under the assay conditions. Dose related values were expressed as a percentage of the positive control values. Sample points were measured as duplicates, errors are expressed as difference from the mean.

The specific cellular 5-³H-Uridine incorporation ratio (%) was calculated in consideration of the results from the DNA synthesis assay. The respective synthesis values (%) were divided by the DNA synthesis coefficient. Raw data are listed in the appendix.

### 2.3.4) Protein Biosynthesis

For the protein synthesis assay cells (HepG2: hepatocellular carcinoma, human; C33-A: cervical carcinoma, human and HT1376: bladder carcinoma, human) were harvested by trypsinisation and seeded at 10,000cells/well in a 96 well plate. After treatment of the cells with the samples at the required concentrations the plate was incubated for 2 hours at 37° C. and 5% CO₂. The cells were pulsed with L-[3,4,5-³H(N)]-leucine, (1 μCi/ml) (Perkin Elmer) during a further incubation period of 24 hours. The cells were washed twice with PBS, lysed with RIPA-buffer and incubated on ice for 15 min. The lysate was transferred in a tube. 250 μl ice cold TCA (20%) was added and the samples were incubated on ice for further 15 min. After centrifugation for 20 min at 10,000 g and 4° C. the supernatant was removed and the pellet was resuspended in 250 μl TCA (5%) and centrifuged again. The received lysates of each sample were resuspended in 250 μl NaOH (0,2N) and heated for 2 min at 50° C. Negative controls t(0) were carried out with the samples without cells.

To detect the incorporated L-[3,4,5-³H(N)]-leucine the samples were transferred in scintillation tubes with scintillation cocktail. The quantification was performed in a Tri-Carb 1900 TR liquid scintillation counter (Packard, USA).

The effect of several concentrations of the sample was measured by determining the amount of radiolabel (dpm) under the assay conditions. Dose related values were expressed as a percentage of the positive control values. Sample points were measured as quadruplicates, errors are expressed as standard deviations.

The specific cellular L-[3,4,5-³H(N)]-leucine incorporation ratio (%) was calculated in consideration of the results from the DNA synthesis assay. The respective synthesis values (%) were divided by the proliferation coefficient. Raw data are listed in the appendix.

### 2.3.5) Apoptosis Assay

HepG2 cells (hepatocellular carcinoma, human) were harvested by trypsinisation and seeded at 10,000 cells/well in 100 μl in a 96 well plate. After 24 h, 20 μl samples and 80 μl of fresh medium (total volume 200 μl) were added. The plate was further incubated for 24 hours and in an additional experiment for 48 hours. After centrifugation of the plate for 10 min at 200×g, the supernatant was discarded and the pellet was lysed for 30 min at room temperature in 200 μl Lysis buffer. The lysate was finally centrifuged for 10 min at 200×g. The solvent controls (=negative controls) were performed with solvent instead of sample. Seeding and incubation were performed in an incubator at 37° C. and 5% CO₂.

The cytoplasmatic histone associated DNA-fragments (mono- and oligonucleosomes) in lysate supernatant were determined in vitro with a Cell Death Detection ELISA^{plus} Kit (Roche, Cat-No: 11 774 425 001) according to the supplier recommendation.

The absorbance was measured with a microplate reader (TECAN Infinite M200) at a wavelength of λ=405 nm and a reference wavelength of λ=490 nm. The specific enrichment factor of mono- and oligonucleosomes released into the cytoplasm was calculated as following:

Enrichment factor=absorbance sample/absorbance of the corresponding negative control

Sample points were measured as duplicates, errors are expressed as difference from the mean.

Raw data are listed in the appendix.

### 2.3.6) LDH-Cytotoxicity Assay (Induction of Necrosis)

LDH-Cytotoxicity assay (Membrane integrity/induction of necrosis): the cytotoxicity was tested on HepG2 cells (hepatocellular carcinoma, human). The cells were seeded in a 96 well plate at 10,000 cells per well over night. 20 μl of the respective samples and solvent controls at the indicated concentrations and 80 μl of fresh medium (total volume 200 μl) were added. Seeding and incubation were performed in an incubator at 37° C. and 5% CO2. After an incubation period of 24 hours, the membrane integrity was measured with a LDH-cytotoxicity Assay Kit (BioVision, #K311-400, CA USA).

The membrane integrity of the cells was evaluated by measuring the activity of cytosolic lactate dehydrogenase (LDH) released into the medium under the influence of samples. The activity of the enzyme was measured by determining the formazan produced from the tetrazolium salt INT as substrate. The quantity of formazan was measured directly by determining the optical density (OD) with a plate reader (TECAN Infinite M200) at a wavelength of λ=490 nm.

For each test concentration, the OD values of the background (assay mixture with samples but without cells) were subtracted from OD measurements with cells. OD₄₉₀-values were transformed into percentage values with cytotoxicity readings of 100% corresponding to measurements of the cells lysed (triton X-100) with solvent only and cytotoxicity readings of 0% corresponding to cells incubated with solvent only. The optical measurements were performed as duplicates. Errors were expressed as difference from the mean.

### 4) SUMMARY

With respect to the DNA-, RNA- and Protein biosynthesis, both Camomile and AMT reveal similar effects: the DNA synthesis is dose dependent suppressed, while up to considerably high doses the RNA synthesis is still intact and the protein biosynthesis is only marginal affected or, if adapted to the amount of proliferating cells, even increased (FIG. 3). This effect is evident for all three cell lines in non cytotoxic dosage ranges and can be interpreted as a sign for cell arrest and possibly differentiation. All tested carcinoma cells, HEPG2 hepatocarcinoma, HT1376 bladder carcinoma and C33A cervix carcinoma showed this phenomenon.

In the subsequently initiated experiments with HEPG2 cells for discrimination between apoptosis and necrosis (indicated by LDH-leakage and loss of cell membrane integrity), after 24 hours we detected only an induction of apoptosis in the highest tested and slightly cytotoxic AMT dosage. Chamomile did not show any effect, neither apoptosis nor cytotoxicity.

Therefore we decided to conduct an additional experiment by incubating the cells for 48 hours. Interestingly, after 48 hours we found an induction of apoptosis by AMT in all tested concentrations in a dose dependent manner, while Camomile showed no effect at all. As Camomile showed considerably strong activity with regard to the depression of DNA-synthesis and increase of protein biosynthesis which correlated very tightly with the concentrations of the Camomile in AMT, we 15 conclude that the cell arresting activity of AMT is most likely caused by its content of Camomile while the induction of apoptosis apparently needs an additional stimulus which is most likely caused by other ingredients of AMT. cl 5) BIBLIOGRAPHY
1 Toshie H., Noriko N. M., Yoshiyuki A., Mittsuhiro N., Toshiro Y., Naohito O. Granulocyte-Macrophage Colony-Stimulating Factor (GM-CSF) Regulates Cytokine Induction by 1,3- -D-Glucan SCG in DBA/2 Mice in vitro J. of IFN and Cytokine Research 24:478-489 (2004)
2 Golenbock D. T., Hampton R. Y., Qureshi N., Takayama K., Raetz C. R. H. (1991) Lipid A-like molecules that antagonize the effect of endotoxins on human monocytes. J. Biol. Chem. 266 (29): 19490-498
3 Garrelds I. M., van Hal P. Th. W., Haakmat R. C., Hoogsteden H. C., Saxena P. R., Zijlstra F. J.: Time dependent production of cytokines and eicosanoides by human monocytic leukaemia U937 cells; effects of glucocorticosteroids. Mediators of Inflammation: 8, 229-235 (1999)
4 Lindl T. (2000) Zell- und Gewebekultur: Einfuhrung in die Grundlagen sowie ausgewahlte Methoden und Anwendungen. 4.uberarb. und erw. Auflage—Heidelberg; Berlin: Spectrum, Akad. Verlag (ISBN 3-8274-0803-2)
5 Decker T. & Lohmann-Matthes M. L. (1988) A quick and simple method for the quantification of lactate dehydrogenase release in measurements of cellular cytotoxicity and tumor necrosis factor activity. J. Immunol Methods 15 (1): 61-69
Abbreviation: Ph. Eur. is Pharmacopoeia Europaea in an amount of 20 ppm or less, in particular 100 ppb or less.

1. Composition comprising an aqueous extract of camomile flowers for the treatment of a proliferative and/or viral condition.

2. The composition according to claim 1, wherein the extract is obtainable by a process comprising purifying an aqueous extract of camomile flowers, preferably camomile tubular flowers.

3. The composition according to claim 2, wherein the camomile flowers are Flores tubiformis.

4. The composition according to claim 2 or 3, wherein the extract contains at least one water-soluble contaminant having lipid groups and the process further comprises the steps of
(i) contacting the composition with a lipophilic component that forms a complex with the contaminant;
(ii) a first removal step of removing material having a particle size larger than the complex formed in step (i); and
(iii) a second removal step of removing the complex formed in step (i).

5. The composition according to claim 4, wherein the contaminant is an endotoxin, preferably selected from the group consisting of fragments of the cell wall or fragments of molecules constituting the cell wall of Gram negative bacteria, more preferably selected from the group consisting of lipopolysaccharides and carbohydrates having protein groups, in particular glycoproteins having at least one lipid chain.

6. The composition according to claim 5, wherein the endotoxin is a carbohydrate having protein groups, in particular a glycoprotein having at least one lipid chain.

7. The composition according to claim 5 or 6, wherein the endotoxin has a molecular weight in excess of 10,000 dalton, more preferably in excess of 5000 dalton, yet more preferably in excess of 1000 dalton and most preferably in excess of 500 dalton.

8. The composition according to any of claims 4 to 7, wherein the lipophilic component is an oil, preferably a fatty oil.

9. The composition according to any of claims 4 to 8, wherein step (ii) comprises microfiltration, preferably using a filter having a pore size of at least 0.05 μm, more preferably having a pore size of from 0.05 to 0.2 μm, most preferablyof 0.1 μm.

10. The composition according to any of claims 4 to 9, wherein step (iii) comprises ultrafiltration, preferably using a filter having a pore size of from 0.001 to 0.02 μm, more preferably from 0.001 to 0.01 μm.

11. The composition according to any of claims 1 to 10, wherein the extract is free or essentially free of endotoxins, preferably the extract contains endotoxins in an amount of 100 EU/ml (endotoxin units per ml according to Ph. Eur.) or less, more preferably 75 EU/ml or less, yet more preferably 50 EU/ml or less, still yet more preferably 25 EU/ml or less, and most preferably 20 EU/ml or less.

12. The composition according to any of claims 1 to 11, wherein the extract is free or essentially free of compounds selected from the group consisting of apigenine, the glycosides of apigenine and essential oils, more preferably free or essentially free of apigenine, the glycosides of apigenine and essential oils, most preferably the extract contains apigenine, the glycosides of apigenine and essential oils in an amount of 20 ppm or less, in particular 100 ppb or less.

13. The composition according to any of claims 1 to 12 additionally comprising ascorbic acid or a salt thereof.

14. The composition according to any of claims 1 to 13 additionally comprising at least one selected from the group consisting of pharmaceutical aids, preferably selected from the group consisting of pharmaceutical agents and pharmaceutical excipients.

15. The composition according to any of claims 1 to 14, characterised in that the treatment is by injection of the composition.

16. The composition according to any of claims 1 to 14, characterised in that the s condition is cancer, in particular liver cancer.

17. Use of the composition as defined in any of claims 1 to 14 for the manufacture of a medicament for the treatment of a proliferative and/or viral condition.

18. The use according to claim 17, wherein the treatment is as defined in claim 15.

19. The use according to claim 18 Wherein the condition is as defined in claim 16.

20. A method for the treatment of a proliferative and/or viral condition, which comprises administering to a human or animal patient in need thereof, in an effective amount, a composition as defined in any of claims 1 to 14.

21. The method according to claim 20, wherein administering is by injection.

22. The method according to claim 21, wherein the composition is an injectable composition.

23. The method according to any of claims 20 to 22, wherein the condition is as defined in claim 16.

24. Composition comprising an aqueous extract of camomile flowers, wherein the extract is free or essentially free of endotoxins.

25. The composition according to claim 24, wherein the extract is of camomile tubular flowers.

26. The composition according to claim 24 or 25, wherein the camomile flowers are Filores tubiformis.

27. The composition according to any of claims 24 to 26, wherein the extract contains endotoxins in an amount of 100 EU/ml (endotoxin units per ml according to Ph. Eur.) or less, more preferably 75 EU/ml or less, yet more preferably 50 EU/ml or less, still yet more preferably 25 EU/ml or less, and most preferably 20 EU/ml or less.

28. The composition according to claim 27, wherein the extract contains endotoxins in an amount of 50 EU/ml or less, preferably 25 EU/ml or less, and most preferably 20 EU/ml or less.

29. The composition according to any of claims 24 to 28, wherein the endotoxins are selected from the group consisting of fragments of the cell wall or fragments of molecules constituting the cell wall of Gram negative bacteria, more preferably selected from the group consisting of lipopolysaccharides and carbohydrates having protein groups, in particular glycoproteins having at least one lipid chain.

30. The composition according to claim 29, wherein the endotoxins are carbohydrates having protein groups, in particular glycoproteins having at least one lipid chain.

31. The composition according to any of claims 24 to 30, wherein the endotoxin has a molecular weight in excess of 10,000 dalton, more preferably in excess of 5000 dalton, yet more preferably in excess of 1000 dalton and most preferably in excess of 500 dalton.

32. The composition according to any of claims 24 to 31, wherein the extract is free or essentially free of compounds selected from the group consisting of apigenine, the glycosides of apigenine and essential oils, more preferably free or essentially free of apigenine, the glycosides of apigenine and essential oils, most preferably the extract contains apigenine, the glycosides of apigenine and essential oils in an amount of 20 ppm or less, in particular 100 ppb or less.

33. The composition according to any of claims 24 to 32 additionally comprising ascorbic acid or a salt thereof.

34. The composition according to any of claims 24 to 33 additionally comprising at least one selected from the group consisting of pharmaceutical aids, preferably selected from the group consisting of pharmaceutical agents and pharmaceutical excipients.
